(19) United States
(12) Patent Application Publication
(10) Pub. No.: US 2020/0246464 A1
(43) Pub. Date: Aug. 6, 2020

(54) PHARMACEUTICAL COMPOSITION

(71) Applicant: Atomis Inc., Kyoto (JP)

(72) Inventors: Daisuke ASARI, Kyoto (JP); Shinji KATO, Kyoto (JP)

(21) Appl. No.: 16/616,954

(22) PCT Filed: Jun. 6, 2018
(86) PCT No.: PCT/JP2018/021694

§ 371 Date: Nov. 25, 2019

(30) Foreign Application Priority Data
Jun. 6, 2017 (JP) 2017112114

(51) Int. Cl. A61K 47/22; A61K 9/00; A61K 33/30; A61K 33/26; A61K 33/06; A61K 33/00

(52) U.S. Cl. CPC A61K 47/22; A61K 9/0014; A61K 9/0053; A61K 33/00; A61K 33/30; A61K 33/26; A61K 33/06; A61K 9/0019

(57) ABSTRACT

An object of the present invention is to provide an excellent pharmaceutical composition. The pharmaceutical composition according to the present invention is a composition for diseases related to immunity, and includes a Metal Organic Framework.

### CROSS-REFERENCE TO RELATED APPLICATIONS

This is a 371 application of International Patent Application Number PCT/JP2018/021694 filed Jun. 6, 2018 claiming priority from Japanese Patent Application Number JP2017-112114 filed Jun. 6, 2017, and the disclosures of which are incorporated herein by reference in their entirety

### TECHNICAL FIELD

The present invention relates to pharmaceutical compositions.

### BACKGROUND ART

Various pharmaceutical compositions have conventionally been developed. On the other hand, a group of materials called Metal Organic Framework (MOF) or Porous Coordination Polymer (PCP) has attracted attention in such fields as gas separation, which are distant from the community of medical science. The MOFs typically form a porous structure by combination of a metal and a multidentate ligand.

### CITATION LIST

### Patent Literature

[Patent Literature 1] WO2004/037895
[Patent Literature 2] WO2009/042802

### Non-Patent Literature

[Non-Patent Literature 1] David Farrusseng, Metal-Organic Frameworks: Applications from Catalysis to Gas Storage, Wiley, 2011
[Non-Patent Literature 2] Yabing He et al. Methane Storage in Metal-Organic Frameworks, Chem Soc Rev., 2014

### SUMMARY OF THE INVENTION

### Technical Problem

An object of the present invention is to provide an excellent pharmaceutical composition.

### Solution to Problem

Some aspects of the present invention are as described below.

[1] A pharmaceutical composition for a disease related to immunity, comprising a Metal Organic Framework (MOF).
[2] The pharmaceutical composition according to [1], further comprising an immune signal transducer.
[3] The pharmaceutical composition according to [2], wherein at least a part of the immune signal transducer is contained in pores of the MOF.
[4] The pharmaceutical composition according to [3], wherein the MOF is configured to decompose in vivo to release at least a part of the immune signal transducer.
[5] The pharmaceutical composition according to any one of [2] to [4], wherein the immune signal transducer is a small molecule having a molecular weight of 1000 or less.
[6] The pharmaceutical composition according to [5], wherein the immune signal transducer is a gas at 25° C. and 100 kPa.
[7] The pharmaceutical composition according to any one of [2] to [6], wherein the immune signal transducer is a factor that is configured to act on keratinocytes, monocytes, lymphocytes, or granulocytes.
[8] The pharmaceutical composition according to any one of [1] to [7], wherein the MOF comprises at least one metal element selected from the group consisting of calcium, magnesium, iron, zinc, aluminum, potassium, and sodium.
[9] The pharmaceutical composition according to any one of [1] to [8], wherein the pharmaceutical composition is configured to be administered by an oral administration, a transdermal administration, and/or a mucosal administration.
[10] The pharmaceutical composition according to any one of claims [1] to [8], wherein the pharmaceutical composition is configured to be administered by an intradermal injection, a subcutaneous injection, or an intramuscular injection.

### Advantageous Effects of Invention

The present invention makes it possible to provide an excellent pharmaceutical composition.

### BRIEF DESCRIPTION OF DRAWINGS

FIG. 1A is a CO adsorption profile of a metal organic framework AP004 [MIL-100 (Fe)].

FIG. 1B is a NO adsorption profile of a metal organic framework AP004 [MIL-100 (Fe)].

FIG. 2 is a NO adsorption profile of a metal organic framework AP104 (BioMIL-3).

FIG. 3 is a graph showing the results of measurement of IL-6 production.

FIG. 4A is a graph showing the results of measurement of IL-6 production.

FIG. 4B is a graph showing the results of measurement of IL-6 production.

FIG. 5 is a graph showing the results of measurement of IL-6 production.

FIG. 6A is a graph showing the results of measurement of TNF-α production.

FIG. 6B is a graph showing the results of measurement of TNF-α production.

FIG. 7 is a graph showing the results of measurement of TNF-α production.

FIG. 8A is a graph showing the results of measurement of IL-1β production.

FIG. 8B is a graph showing the results of measurement of IL-1β production.

FIG. 9 is a graph showing the results of measurement of IL-1β production.

### DESCRIPTION OF EMBODIMENTS

Pharmaceutical compositions according to an embodiment of the present invention are hereinafter described.

The pharmaceutical composition according to the present disclosure is a pharmaceutical composition for diseases related to immunity (hereinafter also referred to as immune diseases). The pharmaceutical composition includes a Metal Organic Framework (MOF). The composition is configured to adjust immune functions.

Examples of the immune diseases targeted by the pharmaceutical composition according to the present disclosure include autoimmune diseases, cancer, allergies, and infectious diseases. Examples of the autoimmune diseases include Alzheimer's disease, Parkinson's disease, Sjogren's syndrome, Passow's disease, Guillain-Barre syndrome, systemic lupus erythematosus, arteriosclerosis, hypertension, type 1 diabetes, myasthenia gravis, rheumatoid arthritis, and osteoporosis. Examples of the Infectious diseases include viral diseases, bacterial diseases, fungal diseases, malaria, Pneumocystis carinii pneumonia, Leishmaniasis, cryptosporidiosis, toxoplasmosis, and trypanosoma infection. The pharmaceutical composition according to the present disclosure can also be used as an immunosuppressant for preventing rejection during organ transplantation.

The Metal Organic Framework (MOF) is formed with a combination of metal(s) and multidentate ligand(s). The mechanism by which the MOF acts on immune diseases is not perfectly clear. The inventors however have attributed the reason to the metal and/or ligand in the MOF interacting with antigens and/or immune cells in some ways. As used herein, the “multidentate ligand” means a ligand that can form two or more coordinate bond.

Any kinds of MOFs can be used in the pharmaceutical composition. Appropriately combining the type and coordination number of the metal ion with the type and topology of the multidentate ligand leads to a MOF with a desired structure. The MOF may be configured to decompose in vivo. The decomposition would expose the metal and the ligand constituting the MOF, by which the MOF might function as a medical compound more efficiently. The MOF can be crystalline or amorphous.

The metal elements in the MOF can be, for example, any elements belonging to alkali metals (Group 1), alkaline earth metals (Group 2), or transition metals (Groups 3 to 12). From the viewpoint of biocompatibility, it is preferable to use at least one metal element selected from the group consisting of calcium, magnesium, iron, zinc, aluminum, potassium, and sodium. However, any metal elements other than these preferable elements can also be used as long as biocompatibility of a MOF as a whole is ensured.

The multidentate ligand in the MOF typically is an organic ligand, examples of which include carboxylate anion and heterocyclic compound. Examples of the carboxylic acid anion include dicarboxylic acid anion and tricarboxylic acid anion. Specific examples include anions of citric acid, malic acid, terephthalic acid, isophthalic acid, trimesic acid, and derivatives thereof. Examples of the heterocyclic compound include bipyridine, imidazole, adenine, and derivatives thereof. Alternatively, the ligand may be an amine compound, a sulfonate anion, or a phosphate anion. The MOF may further contain monodentate ligand(s).

The combination of the metal and the ligand forming the MOF can be appropriately determined according to the expected function and the desired pore size. The MOF may contain two or more types of metal elements, and may contain two or more types of ligands. The MOF can be surface-modified with a polymer or other modifiers.

Specific examples of the MOF include those listed in Table 1 of the Non-Patent Literature 2. Those shown in Tables 1 to 3 below may also be used as the MOF. These are non-limiting lists, and other MOFs can also be used.

TABLE 1

| Name/ | Metal | Ligand |
|---|---|---|
| Abbreviation | (Cation) | (Anion) |
| CPL-1 | Cu | pzdc (2,3-pyrazinedicarboxylic acid), |
| | | pyz (pyrazine) |
| Cu₃(btc)₂ | Cu | BTC (trimesic acid) |
| Zn₂(14bdc)₂(dabco) | Zn | BDC (terephthalic acid), dabco |
| | | (1,4-diazabicyclo[2,2,2]octane) |
| ZIF-8 | Zn | imidazole |
| HKUST-1 | Cu | 1,3,5-benzenetricarboxylic acid |
| Mg₃(C₁₂O₁₄H₁₀) | Mg | citric acid |
| Ca₂(C₈O₁₂H₆) | Ca | malic acid |
| Ca₃(C₁₂O₁₄H₁₀) | Ca | citric acid |
| Ca(C₄O₆H₄) | Ca | malic acid |
| Cu(IPA) | Cu | isophthalic acid |
| MgBDC-1 | Mg | BDC (terephthalic acid) |
| MgDHBDC-1 | Mg | DHBDC (2,5-dihydroxyterephthalic acid) |
| MgOBA-1 | Mg | OBA (4,4′-oxobisbenzoic acid) |
| MgBTC-1 | Mg | BTC (trimesic acid) |
| MgBTB-1 | Mg | BTB (1,3,5-tri(4′-carboxy-4,4′- |
| | | biphenyl)benzene) |
| MgBTB-2 | Mg | BTB (1,3,5-tri(4′-carboxy-4,4′- |
| | | biphenyl)benzene) |
| MgBTB-3 | Mg | BTB (1,3,5-tri(4′-carboxy-4,4′- |
| | | biphenyl)benzene) |
| MgBTB-4 | Mg | BTB (1,3,5-tri(4′-carboxy-4,4′- |
| | | biphenyl)benzene) |
| MgBBC-1 | Mg | BBC (4,4′-4″-benzene-1,3,5-triyl- |
| | | tri-biphenylcarboxylic acid) |
| MIL-100(Fe) | Fe | BTC (trimesic acid) |
| MIL-101 | Fe | BDC (terephthalic acid) |
| MIL-53 | Fe | BDC (terephthalic acid) |
| BioMIL-5 | Zn | azelaic acid |
| CaZol nMOF | Ca | zoledronic acid |
| IRMOF-2 | Zn | o-Br-BDC (o-bromoterephthalic acid) |
| IRMOF-3 | Zn | H₂N-BDC (2-aminoterephthalic acid) |
| IRMOF-4 | Zn | [C₃H₇O]₂-BDC |
| IRMOF-5 | Zn | [C₅H₁₁O]₂-BDC |
| IRMOF-6 | Zn | [C₂H₄]-BDC |
| IRMOF-7 | Zn | 1,4-NDC (1,4-naphthalenedicarboxylic |
| | | acid) |
| IRMOF-8 | Zn | 2,6-NDC (2,6-naphthalenedicarboxylic |
| | | acid) |
| IRMOF-9 | Zn | BPDC (4,4′-biphenyldicarboxylic acid) |
| IRMOF-10 | Zn | BPDC (4,4′-biphenyldicarboxylic acid) |
| IRMOF-11 | Zn | HPDC (tetrahydropyrene-2,7- |
| | | dicarboxylic acid) |
| IRMOF-12 | Zn | HPDC (tetrahydropyrene-2,7- |
| | | dicarboxylic acid) |
| IRMOF-13 | Zn | PDC (pyrene dicarboxylic acid) |
| IRMOF-14 | Zn | PDC (pyrene dicarboxylic acid) |
| IRMOF-15 | Zn | TPDC (terphenyl dicarboxylic acid) |
| IRMOF-16 | Zn | TPDC (terphenyl dicarboxylic acid) |

TABLE 2

| Name/ | Metal | Ligand |
|---|---|---|
| Abbreviation | (Cation) | (Anion) |
| Zn₃(BTC)₂ | Zn | BTC (trimesic acid) |
| Zn₄O(NDC) | Zn | 1,4-NDC (1,4-naphthalene- |
| | | dicarboxylic acid) |
| Mg(Formate) | Mg | formic acid |
| Fe(Formate) | Fe | formic acid |
| Mg(C₆H₄O₆) | Mg | DHBDC (2,5-dihydroxyterephthalic acid) |
| ZnC₂H₄BDC | Zn | [C₂H₄]-BDC |
| MOF-49 | Zn | m-BDC |
| BPR95A2 | Zn | BDC (terephthalic acid) |
| BPR76D5 | Zn | BzPDC |
| BPR68D10 | Zn | BTC (trimesic acid) |
| BPR56E1 | Zn | BDC (terephthalic acid) |
| BPR49B1 | Zn | BDC (terephthalic acid) |
| BPR43G2 | Zn | BDC (terephthalic acid) |
| NO336 | Fe | formic acid |
| NO335 | Fe | formic acid |
| NO333 | Fe | formic acid |
| PCN-14 | Nb | 5,5′-(9,10-anthracenediyl) |
| | | diisophosphate |
| Zn₄BNDC | Zn | BNDC (1,1′-binaphthyl-4,4′- |
| | | dicarboxylic acid) |
| Zn₃(BPDC) | Zn | BPDC (4,4′-biphenyldicarboxylic acid) |
| ZnDBP | Zn | DBP (dibenzyl phosphate) |
| Zn₃(PDC)_{2.5} | Zn | PDC (pyrene dicarboxylic acid) |
| Zn(HPDC) | Zn | HPDC (tetrahydropyrene-2,7-dicarboxylic acid) |
| Zn(NDC) | Zn | 2,6-NDC (2,6-naphthalenedicarboxylic acid) |
| MOF-37 | Zn | 2,6-NDC (2,6-naphthalenedicarboxylic acid) |
| MOF-20 | Zn | 2,6-NDC (2,6-naphthalenedicarboxylic acid) |
| MOF-12 | Zn | ATC (1,3,5,7-adamantanetetracarboxylic acid) |
| Zn(ADC) | Zn | ADC (acetylenedicarboxylic acid) |
| MOF-0 | Zn | BTC (trimesic acid) |
| MOF-2 | Zn | BDC (terephthalic acid) |
| MOF-3 | Zn | BDC (terephthalic acid) |
| MOF-4 | Zn | BTC (trimesic acid) |
| MOF-5 | Zn | BDC (terephthalic acid) |
| MOF-38 | Zn | BTC (trimesic acid) |
| MOF-31 | Zn | ADC (acetylenedicarboxylic acid) |
| MOF-69A | Zn | BPDC (4,4′-biphenyldicarboxylic acid) |
| MOF-69B | Zn | 2,6-NDC (2,6-naphthalenedicarboxylic acid) |
| MOF-33 | Zn | ATB (adamantanetetrabenzoic acid) |
| MOF-36 | Zn | MTB (methanetetrabenzoic acid) |
| MOF-39 | Zn | BTB (1,3,5-tri(4′-carboxy-4,4′- |
| | | biphenyl)benzene) |

TABLE 3

| Name/ | Metal | Ligand |
|---|---|---|
| Abbreviation | (Cation) | (Anion) |
| NO305 | Fe | formic acid |
| NO306A | Fe | formic acid |
| BPR48A2 | Zn | BDC (terephthalic acid) |
| Zn(C₂O₄) | Zn | oxalic acid |
| MOF-48 | Zn | 2,6-NDC |
| | | (2,6-naphthalenedicarboxylic acid) |
| MOF-47 | Zn | BDC(CH₃)₄ |
| Zn₃(BTC)₂ | Zn | BTC (trimesic acid) |
| MOF-n | Zn | BTC (trimesic acid) |
| Zehex | Zn | BTB (1,3,5-tri(4′-carboxy-4,4′- |
| | | biphenyl)benzene) |
| AS16 | Fe | BDC (terephthalic acid) |
| AS27-3 | Fe | BDC (terephthalic acid) |
| AS54-3 | Fe | BPDC (4,4′- |
| | | biphenyldicarboxylic acid) |
| AS61-4 | Fe | m-BDC |
| AS68-7 | Fe | m-BDC |
| Zn₈(ad)₄(PDAC)₆(OH)₂ | Zn | adenine, PDAC (1,4-diphenyl |
| | | diacrylic acid) |
| Zn₈(ad)₄(SBDC)₆(OH)₂ | Zn | adenine, SBDC (4,4′-stilbene |
| | | dicarboxylic acid) |
| Zn₈(ad)₄(BPDC)₆(OH)₂ | Zn | adenine, BPDC |
| Zn₈(ad)₄(NDC)₆(OH)₂ | Zn | adenine, 2,6-NDC |
| M-CPO-27 | Mg | DHBDC (2,5-dihydroxyterephthalic |
| | | acid) |
| bio-MOF-1 | Zn | adenine, BPDC |
| UMCM-1 | Zn | BTB (1,3,5-tri(4′-carboxy-4,4′- |
| | | biphenyl)benzene) |
| UMCM-2 | Zn | BTB (1,3,5-tri(4′-carboxy-4,4′- |
| | | biphenyl)benzene) |
| MOF-210 | Zn | BTE (4,4′,4″-[benzene-1,3,5- |
| | | triyl-tris (ethyne-2, 1-diyl)] |
| | | tribenzoic acid), BPDC |
| bio-MOF-100 | Zn | adenine, BPDC |
| NU-110E | Cu | J. Am. Chem. Soc. 2012, 134, |
| | | 15016-15021 |
| CD-MOF-1 | K | γ-CD (γ-cyclodextrin) |
| porph@MOM-4 | Fe | porphyrin, BTC |
| porph@MOM-8 | Mg | porphyrin, BTC |
| porph@MOM-9 | Zn | porphyrin, BTC |
| ZnPO-MOF | Zn | metalloporphyrin pyridyl, TCPB |
| | | (1,2,4,5-Tetrakis(4- |
| | | carboxyphenyl)benzene) |
| Uio-66 | Fe | DCBDT (1,4-dicarboxylbenzene-2,3- |
| | | dithiolate) |
| Mg(H₂gal) | Mg | caustic acid (3,4,5-trihydroxybenzoic |
| | | acid) |

Particularly preferable MOFs include the followings.

TABLE 4

| Abbreviation | Metal | Ligand |
|---|---|---|
| AP008 ZIF-8 | Zn²⁺ | 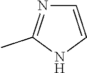 |
| | | 2-methylimidazole |
| AP004 MIL-100(Fe) | Fe³⁺ | 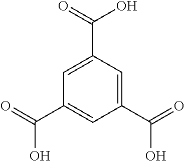 [SMILES: O=C(O)c1cc(C(=O)O)cc(C(=O)O)c1] |
| | | 1,3,5-benzenetricarboxylic |
| | | acid |
| AP006 Al(Fumarate) | Al³⁺ | 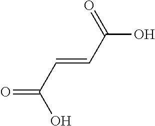 |
| | | fumaric acid |
| AP005 MIL-53(Al) | Al³⁺ | 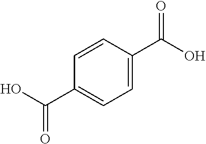 [SMILES: O=C(O)c1ccc(C(=O)O)cc1] |
| | | 1,4-benzenedicarboxylic acid |

TABLE 5

| Abbreviation | Metal | Ligand |
|---|---|---|
| AP101 | Ca²⁺ | 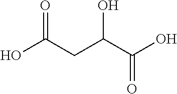 |
| | | DL-malic acid |
| AP104 BioMIL-3 | Ca²⁺ | 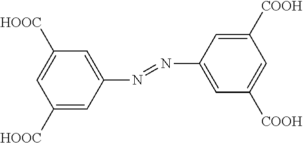 [SMILES: O=C(O)c1cc(/N=N/c2cc(C(=O)O)cc(C(=O)O)c2)cc(C(=O)O)c1] |
| | | 3,3′,5,5′-azobenzenetetracarboxylic acid |
| AP009 Mg(Formate) | Mg²⁺ | 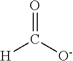 |
| | | formic acid |
| AP014 | La³⁺ | 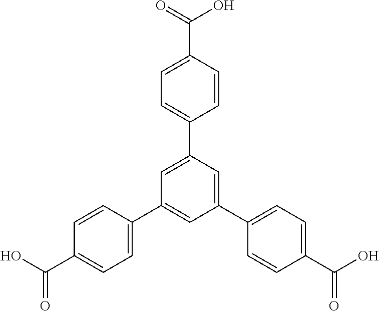 [SMILES: O=C(O)c1ccc(-c2cc(-c3ccc(C(=O)O)cc3)cc(-c3ccc(C(=O)O)cc3)c2)cc1] |
| | | BTB |

TABLE 6

| Abbreviation | Metal | Ligand |
|---|---|---|
| AP102 | Ca²⁺ | 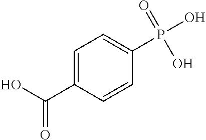 [SMILES: O=C(O)c1ccc(P(=O)(O)O)cc1] |
| | | 4-phosphonobenzoic acid |
| AP103 | Ca²⁺ | 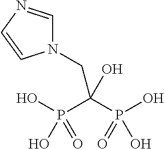 [SMILES: O=P(O)(O)C(O)(Cn1ccnc1)P(=O)(O)O] |
| | | zoledronic acid monohydrate |
| AP105 | Ca²⁺ | 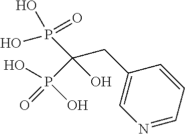 [SMILES: O=P(O)(O)C(O)(Cc1cccnc1)P(=O)(O)O] |
| | | risedronic acid |

TABLE 7

| Abbreviation | Metal | Ligand |
|---|---|---|
| AP107 | Al³⁺ | 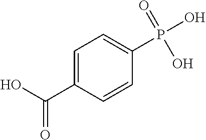 [SMILES: O=C(O)c1ccc(P(=O)(O)O)cc1] |
| | | 4-phosphonobenzoic acid |
| AP106 | mg²⁺ | 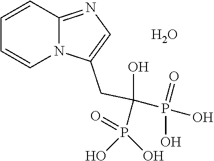 |
| | | minodronic acid monohydrate |
| AP108 | Ca²⁺ | 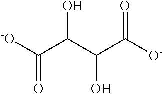 |
| | | tartaric acid |
| AP015 | Ca²⁺ | 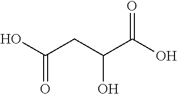 |
| | | malic acid |

TABLE 8

| Abbreviation | Metal | Ligand |
|---|---|---|
| AP001 | Cu²⁺ | 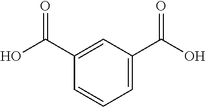 [SMILES: O=C(O)c1cccc(C(=O)O)c1] |
| | | isophthalic acid |
| AP003 Fe-BTC | Fe³⁺ | 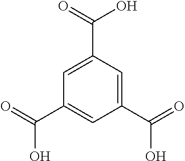 [SMILES: O=C(O)c1cc(C(=O)O)cc(C(=O)O)c1] |
| | | 1,3,5-benzenetricarboxylic acid |
| Ni-MOF-74 | Ni²⁺ | 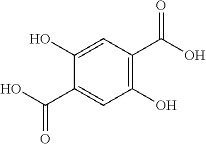 [SMILES: O=C(O)c1cc(O)c(C(=O)O)cc1O] |
| | | 2,5-dihydroxyterephthalic acid |
| Co-MOF-74 | Co²⁺ | 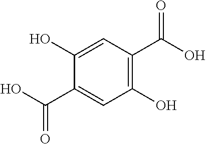 [SMILES: O=C(O)c1cc(O)c(C(=O)O)cc1O] |
| | | 2,5-dihydroxyterephthalic acid |

TABLE 9

| Abbreviation | Metal | Ligand |
|---|---|---|
| MIL-88-A | Fe²⁺ | 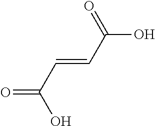 |
| | | fumaric acid |
| MIL-88-B | Fe²⁺ | 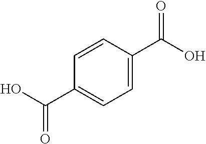 [SMILES: O=C(O)c1ccc(C(=O)O)cc1] |
| | | terephthalic acid |

Only one type of MOF may be used, or two or more types thereof may be used in combination. The content of the MOF in the pharmaceutical composition is, for example, 1×10⁻⁷ mass % or more, preferably 1×10⁻⁶ mass % or more, and more preferably 5×10⁻⁶ mass % or more.

The pharmaceutical composition according to one embodiment of the present invention may further contain an immune signal transducer. Adopting such a configuration can further enhance the effect of administering the pharmaceutical composition. As used herein, the “immune signal transducer” means any substance used for transmitting an immune signal for inducing activation and/or differentiation of immune cells. The immune signal transducer may be, for example, cytokines such as interleukins, chemokines, interferons, hematopoietic factors, cell growth factors, or cell necrosis factors, or may be small molecules such as gas molecules that will be described later. As used herein, the “small molecule” means a molecule having a molecular weight of 1000 or less.

The immune signal transducer is, for example, a factor that is configured to act on lymphocytes (T cells, B cells, NK cells, etc.), monocytes (macrophages, Langerhans cells, dendritic cells, etc.), granulocytes (neutrophils, eosinophils, basophils, etc.) and/or keratinocytes. The immune signal transducer is, for example, a factor that is configured to induce differentiation of helper T cells, which are a type of lymphocyte, into various lineages such as Th1 cells, Th2 cells, Treg cells, Th17 cells, Tfh cells, or memory T cells. When the immune signal transducer induces Th1 cells, the pharmaceutical composition according to the present invention can be used, for example, as a medicine for cancer or infectious diseases. When the immune signal transducer induces Th2 cells, the pharmaceutical composition according to the present invention can be used, for example, as a medicine for infectious diseases or lifestyle-related diseases. When the immune signal transducer induces Treg cells, the pharmaceutical composition according to the present invention can be used, for example, as a medicine for allergy or for organ transplants. When the immune signal transducer induces Th17 cells, the pharmaceutical composition according to the present invention can be used, for example, as a medicine for infectious diseases. When the immune signal transducer induces Tfh cells, the pharmaceutical composition according to the present invention can be used, for example, as a medicine for infectious diseases. When the immune signal transducer induces memory T cells, the pharmaceutical composition according to the present invention can be used, for example, as a medicine for infectious diseases or cancer.

It is preferable that at least a part of the immune signal transducer is contained in the pores of the MOF. This allows for more stable and quantitative administration of the immune signal transducer. In such a case, the other part of the immune signal transducer may be attached to the surface of the MOF. Alternatively, most of the immune signal transducer may be contained in the pores of the MOF.

When at least a part of the immune signal transducer is contained in the pores of the MOF, it is preferable that the MOF has an irreversible adsorption/desorption profile. That is, the MOF preferably retains a larger amount of guest molecules at the time of desorption than the amount of guest molecules at the time of adsorption at the same pressure. It is particularly preferable that the residual amount of the guest molecule in the MOF is non-zero after performing the adsorption process from a vacuum state to a pressurized state and then performing the desorption process from the pressurized state to the vacuum state. This enables easier retention of the immune signal transducer in the pores of the MOF under the condition of low pressure (e.g. at atmospheric pressure).

When at least a part of the immune signal transducer is contained in the pores of the MOF, it is also preferable that the MOF is configured to decompose in vivo to release at least a part of the immune signal transducer. This allows finer adjustment of the dose and the release rate of the immune signal transducer. The decomposition may also induce more exposure of the metal and the ligand of the MOF, thereby further enhancing the function of the MOF as a medical compound.

As described above, the immune signal transducer can be a small molecule. This makes it easier to include at least a part of the immune signal transducer in the pores of the MOF. As used herein, again, the “small molecule” means a molecule having a molecular weight of 1000 or less.

More preferably, the immune signal transducer is a gas under the condition of 25° C. and 100 kPa (i.e. SATP). This makes it still easier to include at least a part of the immune signal transducer in the pores of the MOF.

In recent years, it has been becoming clear that small molecules such as gas molecules function as immune signal transducers. For example, gas molecules such as nitric oxide, carbon monoxide, carbon dioxide, hydrogen sulfide, or methane have been shown to act on immunocompetent cells. However, there have been no method for stably and quantitatively administering small molecules such as gas molecules into a living body, and a person skilled in the art has not tried it yet because of its anticipated difficulty. The present inventors have however found that small molecules such as gas molecules can be stably and quantitatively administered in vivo by using small molecules such as gas molecules along with the MOF.

There are no particular limitations on the small molecules or gas molecules used as immune signal transducers. Examples of such an immune signal transducer include compounds shown in Table 10 below. These are non-limiting lists, and other small molecules or gas molecules may be used.

TABLE 10

| | |
|---|---|
| Diatomic molecules | Nitrogen, oxygen, hydrogen, fluorine, chlorine, |
| | bromine, iodine |
| Noble gases | Helium, neon, argon, krypton, xenon, radon |
| Carbon oxides | Carbon monoxide, carbon dioxide |
| Nitrogen compounds | Ammonia, nitric oxide, nitrogen dioxide, |
| | dinitrogen monoxide, dinitrogen tetroxide, |
| | dinitrogen trioxide, dinitrogen pentoxide, |
| | dimethylamine, trimethylamine |
| Sulfur compounds | Sulfur dioxide, hydrogen sulfide, methanethiol, |
| | dimethyl sulfide |
| Alkanes | Methane, ethane, propane, butane, |
| | halogenated methane |
| Alkenes | Ethylene, propylene, butadiene |
| Alkynes | Acetylene |
| Alcohols | Methanol, ethanol, propanol |
| Aldehydes | Formaldehyde, acetaldehyde |
| Carboxylic acids | Formic acid, acetic acid, citric acid, malic acid |
| Ethers | Dimethyl ether, diethyl ether |
| Aromatic compounds | Benzene, toluene |
| Others | Water, bioactive substances |

Only one type of immune signal transducer may be used, or two or more types thereof may be used in combination. The content of the immune signal transducer in the pharmaceutical composition is, for example, in the range of 1×10⁻⁷ to 40% by mass, preferably in the range of 1×10⁻⁶ to 30% by mass, and more preferably in the range of 5×10⁻⁵ to 25 mass %.

Any methods can be used for introducing the immune signal transducer into the pores of the MOF. For example, a solution or dispersion of a MOF may be mixed with a solution or dispersion of an immune signal transducer. Alternatively, a solid MOF may be exposed to an immune signal transducer or a solution or dispersion thereof. When the immune signal transducer is a gas, the MOF may be simply exposed to the gas.

The pharmaceutical composition according to one embodiment of the present invention may further contain other component(s) than the MOF. For example, the pharmaceutical composition may further contain immunostimulant(s) such as a TLR ligand, an RLR ligand, an NLR ligand, or a cyclic dinucleotide.

The pharmaceutical composition according to one embodiment of the present invention can be dissolved or dispersed in a solvent when in use. Examples of such solvents include physiological saline, phosphate buffered saline (PBS), glycerin, propylene glycol, polyethylene glycol, fats, or oils.

The pharmaceutical composition according to the present invention can be administered to a subject by any method. As used herein, the “subject” refers to any animal whose immune response can be induced upon administration of pharmaceutical composition in the practical stage. The animal typically is a mammal including humans, such as mice, rats, dogs, cats, rabbits, horses, cow, sheep, pig, goat, monkey, chimpanzee, ferret, mole, etc. A particularly preferred subject is a human.

The pharmaceutical composition according to one embodiment of the present invention may be configured to be administered, for example, by an oral, transdermal, and/or mucosal administration.

In the case of oral administration, the pharmaceutical composition may be any formulation commonly used for oral administration. For example, tablets (including orally disintegrating tablets), pills, powders, fine granules, granules, chewable tablets, capsules, jellies, extracts, elixirs, solutions, suspensions, spirits, syrups, soaking agents, decoction, tincture, aromatic liquid, limonade, or flow extract can be used. The classification, definition, properties, and production method of these compositions are well known in the art, and can be found, for example, in the Japanese Pharmacopoeia 16th edition.

In the case of transdermal administration, the pharmaceutical composition may be any formulation commonly used for transdermal administration. For example, liquid for external use such as liniments or lotions, external sprays such as aerosols, ointments, plasters, creams, gels, or patches such as tapes or poultices can be used. The classification, definition, properties, and production method of these compositions are well known in the art, and can be found, for example, in the Japanese Pharmacopoeia 16th edition.

In the case of mucosal administration, the pharmaceutical composition may be any formulation commonly used for mucosal administration such as sublingual, nasal, buccal, rectal or vaginal administration. For example, semi-solid preparations such as gel (jelly), cream, ointment, or plasters, liquid preparations, solid preparations such as powders, fine granules, granules, films, tablets, or orally disintegrating tablets, sprays for mucous membranes such as aerosols, or inhalants can be used. The classification, definition, properties, and production method of these compositions are well known in the art, and can be found, for example, in the Japanese Pharmacopoeia 16th edition.

The pharmaceutical composition according to one aspect of the present invention is configured to be administered, for example, by intradermal injection, subcutaneous injection, or intramuscular injection. In the case of intradermal, subcutaneous, or intramuscular administration, the composition may be in a form that has a certain fluidity that can be administered by injection, such as a liquid, suspension, cream, and the like. The classification, definition, properties, and production method of these compositions are well known in the art, and can be found, for example, in the Japanese Pharmacopoeia 16th edition.

The pharmaceutical composition may further contain additive(s) if necessary. The additives can be selected depending, for example, upon main component of the base, compatibility with the MOF, or the intended dosage regimen. Examples of the additives include skin permeability enhancers, isotonic agents, antiseptic/disinfectants, antioxidants, solubilizers, solubilizing agents, suspending agents, fillers, pH adjusters, stabilizers, absorption enhancers, release rate controllers, colorants, plasticizers, adhesives, or their combinations.

### EXAMPLES

### Preparation of Sample Solutions

### Comparative Example 1

Physiological saline (Otsuka Normal Saline, Otsuka Pharmaceutical) itself was used as a sample solution.

### Example 1

1 mg of ZIF-8 (Basolite Z1200, Sigma-Aldrich) was added to and mixed with 10 mL of physiological saline (Otsuka Normal Saline, Otsuka Pharmaceutical) to obtain a sample solution.

### Example 2

NO (nitrogen monoxide, Kyoto Teijin) was bubbled in 100 mL of physiological saline (Otsuka Normal Saline, Otsuka Pharmaceutical) at room temperature for 6 hours to prepare NO saturated physiological saline. To 10 mL of the obtained solution was added 1 mg of ZIF-8 (Basolite Z1200, Sigma-Aldrich), and these were mixed to provide a sample solution.

The above configuration is summarized in Table 11 below.

TABLE 11

| | MOF | | | | Immune Signal Transducer | |
|---|---|---|---|---|---|---|
| | | Concentration | Solvent | | | Concentration |
| | Name | [μg/mL] | Name | Amount [μL] | Name | [mM] |
| Comp. Ex. 1 | — | — | Physiological | 100 | — | — |
| | | | saline | | | |
| Example | ZIF-8 | 100 | Physiological | 100 | — | — |
| 1 | | | saline | | | |
| Example | ZIF-8 | 100 | Physiological | 100 | NO | 1.8 |
| 2 | | | saline | | | |

### Examples 3 to 31

Sample solutions were prepared in the same manner as in Example 2 except that the substances shown in Table 12 below were used instead of NO as immune signal transducers.

TABLE 12

| | | | | | Immune Signal | |
|---|---|---|---|---|---|---|
| | MOF | | Solvent | | Transducer | |
| | | Concentration | | Amount | | Concentration |
| | Name | [μg/mL] | Name | [μL] | Name | [mM] |
| Example 2 | ZIF-8 | 100 | Physiological saline | 100 | NO | Saturated |
| Example 3 | ZIF-8 | 100 | Physiological saline | 100 | CO | Saturated |
| Example 4 | ZIF-8 | 100 | Physiological saline | 100 | CO₂ | Saturated |
| Example 5 | ZIF-8 | 100 | Physiological saline | 100 | N₂ | Saturated |
| Example 6 | ZIF-8 | 100 | Physiological saline | 100 | O₂ | Saturated |
| Example 7 | ZIF-8 | 100 | Physiological saline | 100 | H₂ | Saturated |
| Example 8 | ZIF-8 | 100 | Physiological saline | 100 | H₂S | Saturated |
| Example 9 | ZIF-8 | 100 | Physiological saline | 100 | S₂O | Saturated |
| Example 10 | ZIF-8 | 100 | Physiological saline | 100 | CH₄ | Saturated |
| Example 11 | ZIF-8 | 100 | Physiological saline | 100 | C₂H₆ | Saturated |
| Example 12 | ZIF-8 | 100 | Physiological saline | 100 | C₃H₈ | Saturated |
| Example 13 | ZIF-8 | 100 | Physiological saline | 100 | C₄H₁₀ | Saturated |
| Example 14 | ZIF-8 | 100 | Physiological saline | 100 | C₂H₄ | Saturated |
| Example 15 | ZIF-8 | 100 | Physiological saline | 100 | C₃H₆ | Saturated |
| Example 16 | ZIF-8 | 100 | Physiological saline | 100 | C₂H₄ | Saturated |
| Example 17 | ZIF-8 | 100 | Physiological saline | 100 | CH₃NH₂ | Saturated |
| Example 18 | ZIF-8 | 100 | Physiological saline | 100 | (CH₃)₂NH | Saturated |
| Example 19 | ZIF-8 | 100 | Physiological saline | 100 | NH₃ | Saturated |
| Example 20 | ZIF-8 | 100 | Physiological saline | 100 | CH₃SH | Saturated |
| Example 21 | ZIF-8 | 100 | Physiological saline | 100 | (CH₃)₃N | Saturated |
| Example 22 | ZIF-8 | 100 | Physiological saline | 100 | CH₃Cl | Saturated |
| Example 23 | ZIF-8 | 100 | Physiological saline | 100 | CH₃Br | Saturated |
| Example 24 | ZIF-8 | 100 | Physiological saline | 100 | He | Saturated |
| Example 25 | ZIF-8 | 100 | Physiological saline | 100 | F₂ | Saturated |
| Example 26 | ZIF-8 | 100 | Physiological saline | 100 | Ne | Saturated |
| Example 27 | ZIF-8 | 100 | Physiological saline | 100 | Cl₂ | Saturated |
| Example 28 | ZIF-8 | 100 | Physiological saline | 100 | Ar | Saturated |
| Example 29 | ZIF-8 | 100 | Physiological saline | 100 | Kr | Saturated |
| Example 30 | ZIF-8 | 100 | Physiological saline | 100 | Xe | Saturated |
| Example 31 | ZIF-8 | 100 | Physiological saline | 100 | Rn | Saturated |

### Examples 32-141

Sample solutions were prepared in the same manner as in Example 2 except that the substances shown in Table 13 to 15 below were used instead of ZIF-8 as MOFs. Abbreviations in Tables 13 to 15 are the same as those described in Tables 1 to 3, respectively.

TABLE 13

| | | | | | Immune Signal | |
|---|---|---|---|---|---|---|
| | MOF | | Solvent | | Transducer | |
| | | Concentration | | Amount | | Concentration |
| | Name | [μg/mL] | Name | [μL] | Name | [mM] |
| Example 2 | ZIF-8 | 100 | Physiological saline | 100 | NO | Saturated |
| Example 32 | CPL-1 | 100 | Physiological saline | 100 | NO | Saturated |
| Example 33 | Cu₃(btc)₂ | 100 | Physiological saline | 100 | NO | Saturated |
| Example 34 | Zn₂(14bdc)₂(dabco) | 100 | Physiological saline | 100 | NO | Saturated |
| Example 35 | ZIF-8 | 100 | Physiological saline | 100 | NO | Saturated |
| Example 36 | HKUST-1 | 100 | Physiological saline | 100 | NO | Saturated |
| Example 37 | Mg₃(C₁₂O₁₄H₁₀) | 100 | Physiological saline | 100 | NO | Saturated |
| Example 38 | Ca₂(C₈O₁₂H₆) | 100 | Physiological saline | 100 | NO | Saturated |
| Example 39 | Ca₃(C₁₂O₁₄H₁₀) | 100 | Physiological saline | 100 | NO | Saturated |
| Example 40 | Ca(C₄O₆H₄) | 100 | Physiological saline | 100 | NO | Saturated |
| Example 41 | Cu(IPA) | 100 | Physiological saline | 100 | NO | Saturated |
| Example 42 | MgBDC-1 | 100 | Physiological saline | 100 | NO | Saturated |
| Example 43 | MgDHBDC-1 | 100 | Physiological saline | 100 | NO | Saturated |
| Example 44 | MgOBA-1 | 100 | Physiological saline | 100 | NO | Saturated |
| Example 45 | MgBTC-1 | 100 | Physiological saline | 100 | NO | Saturated |
| Example 46 | MgBTB-1 | 100 | Physiological saline | 100 | NO | Saturated |
| Example 47 | MgBTB-2 | 100 | Physiological saline | 100 | NO | Saturated |
| Example 48 | MgBTB-3 | 100 | Physiological saline | 100 | NO | Saturated |
| Example 49 | MgBTB-4 | 100 | Physiological saline | 100 | NO | Saturated |
| Example 50 | MgBBC-1 | 100 | Physiological saline | 100 | NO | Saturated |
| Example 51 | MIL-100(Fe) | 100 | Physiological saline | 100 | NO | Saturated |
| Example 52 | MIL-101 | 100 | Physiological saline | 100 | NO | Saturated |
| Example 53 | MIL-53 | 100 | Physiological saline | 100 | NO | Saturated |
| Example 54 | BioMIL-5 | 100 | Physiological saline | 100 | NO | Saturated |
| Example 55 | CaZol nMOF | 100 | Physiological saline | 100 | NO | Saturated |
| Example 56 | IRMOF-2 | 100 | Physiological saline | 100 | NO | Saturated |
| Example 57 | IRMOF-3 | 100 | Physiological saline | 100 | NO | Saturated |
| Example 58 | IRMOF-4 | 100 | Physiological saline | 100 | NO | Saturated |
| Example 59 | IRMOF-5 | 100 | Physiological saline | 100 | NO | Saturated |
| Example 60 | IRMOF-6 | 100 | Physiological saline | 100 | NO | Saturated |
| Example 61 | IRMOF-7 | 100 | Physiological saline | 100 | NO | Saturated |
| Example 62 | IRMOF-8 | 100 | Physiological saline | 100 | NO | Saturated |
| Example 63 | IRMOF-9 | 100 | Physiological saline | 100 | NO | Saturated |
| Example 64 | IRMOF-10 | 100 | Physiological saline | 100 | NO | Saturated |
| Example 65 | IRMOF-11 | 100 | Physiological saline | 100 | NO | Saturated |
| Example 66 | IRMOF-12 | 100 | Physiological saline | 100 | NO | Saturated |
| Example 67 | IRMOF-13 | 100 | Physiological saline | 100 | NO | Saturated |
| Example 68 | IRMOF-14 | 100 | Physiological saline | 100 | NO | Saturated |
| Example 69 | IRMOF-15 | 100 | Physiological saline | 100 | NO | Saturated |
| Example 70 | IRMOF-16 | 100 | Physiological saline | 100 | NO | Saturated |

TABLE 14

| | MOF | | Solvent | | Immune Signal Transducer | |
|---|---|---|---|---|---|---|
| | | Concentration | | Amount | | Concentration |
| | Name | [μg/mL] | Name | [μL] | Name | [mM] |
| Example 71 | Zn₃(BTC)₂ | 100 | Physiological saline | 100 | NO | Saturated |
| Example 72 | Zn₄O(NDC) | 100 | Physiological saline | 100 | NO | Saturated |
| Example 73 | Mg(Formate) | 100 | Physiological saline | 100 | NO | Saturated |
| Example 74 | Fe(Formate) | 100 | Physiological saline | 100 | NO | Saturated |
| Example 75 | Mg(C₆H₄O₆) | 100 | Physiological saline | 100 | NO | Saturated |
| Example 76 | ZnC₂H₄BDC | 100 | Physiological saline | 100 | NO | Saturated |
| Example 77 | MOF-49 | 100 | Physiological saline | 100 | NO | Saturated |
| Example 78 | BPR95A2 | 100 | Physiological saline | 100 | NO | Saturated |
| Example 79 | BPR76D5 | 100 | Physiological saline | 100 | NO | Saturated |
| Example 80 | BPR68D10 | 100 | Physiological saline | 100 | NO | Saturated |
| Example 81 | BPR56E1 | 100 | Physiological saline | 100 | NO | Saturated |
| Example 82 | BPR49B1 | 100 | Physiological saline | 100 | NO | Saturated |
| Example 83 | BPR43G2 | 100 | Physiological saline | 100 | NO | Saturated |
| Example 84 | NO336 | 100 | Physiological saline | 100 | NO | Saturated |
| Example 85 | NO335 | 100 | Physiological saline | 100 | NO | Saturated |
| Example 86 | NO333 | 100 | Physiological saline | 100 | NO | Saturated |
| Example 87 | PCN-14 | 100 | Physiological saline | 100 | NO | Saturated |
| Example 88 | Zn₄BNDC | 100 | Physiological saline | 100 | NO | Saturated |
| Example 89 | Zn₃(BPDC) | 100 | Physiological saline | 100 | NO | Saturated |
| Example 90 | ZnDBP | 100 | Physiological saline | 100 | NO | Saturated |
| Example 91 | Zn₃(PDC)_{2.5} | 100 | Physiological saline | 100 | NO | Saturated |
| Example 92 | Zn(HPDC) | 100 | Physiological saline | 100 | NO | Saturated |
| Example 93 | Zn(NDC) | 100 | Physiological saline | 100 | NO | Saturated |
| Example 94 | MOF-37 | 100 | Physiological saline | 100 | NO | Saturated |
| Example 95 | MOF-20 | 100 | Physiological saline | 100 | NO | Saturated |
| Example 96 | MOF-12 | 100 | Physiological saline | 100 | NO | Saturated |
| Example 97 | Zn(ADC) | 100 | Physiological saline | 100 | NO | Saturated |
| Example 98 | MOF-0 | 100 | Physiological saline | 100 | NO | Saturated |
| Example 99 | MOF-2 | 100 | Physiological saline | 100 | NO | Saturated |
| Example 100 | MOF-3 | 100 | Physiological saline | 100 | NO | Saturated |
| Example 101 | MOF-4 | 100 | Physiological saline | 100 | NO | Saturated |
| Example 102 | MOF-5 | 100 | Physiological saline | 100 | NO | Saturated |
| Example 103 | MOF-38 | 100 | Physiological saline | 100 | NO | Saturated |
| Example 104 | MOF-31 | 100 | Physiological saline | 100 | NO | Saturated |
| Example 105 | MOF-69A | 100 | Physiological saline | 100 | NO | Saturated |
| Example 106 | MOF-69B | 100 | Physiological saline | 100 | NO | Saturated |
| Example 107 | MOF-33 | 100 | Physiological saline | 100 | NO | Saturated |
| Example 108 | MOF-36 | 100 | Physiological saline | 100 | NO | Saturated |
| Example 109 | MOF-39 | 100 | Physiological saline | 100 | NO | Saturated |

TABLE 15

| | MOF | | Solvent | | Immune Signal Transducer | |
|---|---|---|---|---|---|---|
| | | Concentration | | Amount | | Concentration |
| | Name | [μg/mL] | Name | [μL] | Name | [mM] |
| Example 110 | NO305 | 100 | Physiological saline | 100 | NO | Saturated |
| Example 111 | NO306A | 100 | Physiological saline | 100 | NO | Saturated |
| Example 112 | BPR48A2 | 100 | Physiological saline | 100 | NO | Saturated |
| Example 113 | Zn(C₂O₄) | 100 | Physiological saline | 100 | NO | Saturated |
| Example 114 | MOF-48 | 100 | Physiological saline | 100 | NO | Saturated |
| Example 115 | MOF-47 | 100 | Physiological saline | 100 | NO | Saturated |
| Example 116 | Zn₃(BTC)₂ | 100 | Physiological saline | 100 | NO | Saturated |
| Example 117 | MOF-n | 100 | Physiological saline | 100 | NO | Saturated |
| Example 118 | Zehex | 100 | Physiological saline | 100 | NO | Saturated |
| Example 119 | AS16 | 100 | Physiological saline | 100 | NO | Saturated |
| Example 120 | AS27-3 | 100 | Physiological saline | 100 | NO | Saturated |
| Example 121 | AS54-3 | 100 | Physiological saline | 100 | NO | Saturated |
| Example 122 | AS61-4 | 100 | Physiological saline | 100 | NO | Saturated |
| Example 123 | AS68-7 | 100 | Physiological saline | 100 | NO | Saturated |
| Example 124 | Zn₈(ad)₄(PDAC)₆(OH)₂ | 100 | Physiological saline | 100 | NO | Saturated |
| Example 125 | Zn₈(ad)₄(SBDC)₆(OH)₂ | 100 | Physiological saline | 100 | NO | Saturated |
| Example 126 | Zn₈(ad)₄(BPDC)₆(OH)₂ | 100 | Physiological saline | 100 | NO | Saturated |
| Example 127 | Zn₈(ad)₄(NDC)₆(OH)₂ | 100 | Physiological saline | 100 | NO | Saturated |
| Example 128 | M-CPO-27 | 100 | Physiological saline | 100 | NO | Saturated |
| Example 129 | bio-MOF-1 | 100 | Physiological saline | 100 | NO | Saturated |
| Example 130 | UMCM-1 | 100 | Physiological saline | 100 | NO | Saturated |
| Example 131 | UMCM-2 | 100 | Physiological saline | 100 | NO | Saturated |
| Example 132 | MOF-210 | 100 | Physiological saline | 100 | NO | Saturated |
| Example 133 | bio-MOF-100 | 100 | Physiological saline | 100 | NO | Saturated |
| Example 134 | NU-110E | 100 | Physiological saline | 100 | NO | Saturated |
| Example 135 | CD-MOF-1 | 100 | Physiological saline | 100 | NO | Saturated |
| Example 136 | porph@MOM-4 | 100 | Physiological saline | 100 | NO | Saturated |
| Example 137 | porph@MOM-8 | 100 | Physiological saline | 100 | NO | Saturated |
| Example 138 | porph@MOM-9 | 100 | Physiological saline | 100 | NO | Saturated |
| Example 139 | ZnPO-MOF | 100 | Physiological saline | 100 | NO | Saturated |
| Example 140 | Uio-66 | 100 | Physiological saline | 100 | NO | Saturated |
| Example 141 | Mg(H₂gal) | 100 | Physiological saline | 100 | NO | Saturated |

[Collection of Intraperitoneal Cells (PEC Cells)]

A mouse was intraperitoneally administered with 2 mL of 4 wt % thioglycolic acid solution, and cells in its peritoneal cavity were taken out 3 days later. The collected cells were then washed with PBS (Phosphate Buffered Saline).

[Stimulation by Sample Solutions]

PEC cells were dispensed in a 24-well plate at 1×10⁶ cells/well, and each sample was added and incubated for 24 hours.

[Cytokine Measurement]

50 μL/well of the supernatant of the cell culture was used for an evaluation by an ELISA kit (Quantikine ELISA kit, R&D Systems) that corresponds to each cytokine (TNF-α, IL-6, IFN-γ, IL-12p40, IL-10) to be monitored. The results are summarized in Table 16 below.

TABLE 16

| | TNF-α | IL-6 | IL-10 | IL-12p40 | IFN-g |
|---|---|---|---|---|---|
| Comp. Ex. 1 | − | − | − | − | − |
| Example 1 | + | + | − | − | − |
| Example 2 | ++ | ++ | − | + | + |

(−): Less than twice the amount of cytokine released in Comparative Example 1
(+): Between twice and three times the amount of cytokine released in Comparative Example 1
(++): Three or more times the amount of cytokine released in Comparative Example 1

[Synthesis of MOFs]

The MOFs shown in Tables 4 to 9 were prepared. Known substances among them were synthesized according to literature methods. The unreported substances were synthesized by hydrothermal treatment of the corresponding metal nitrate and the ligand in the presence of DMF.

[Evaluation of Adsorption Properties of MOFs]

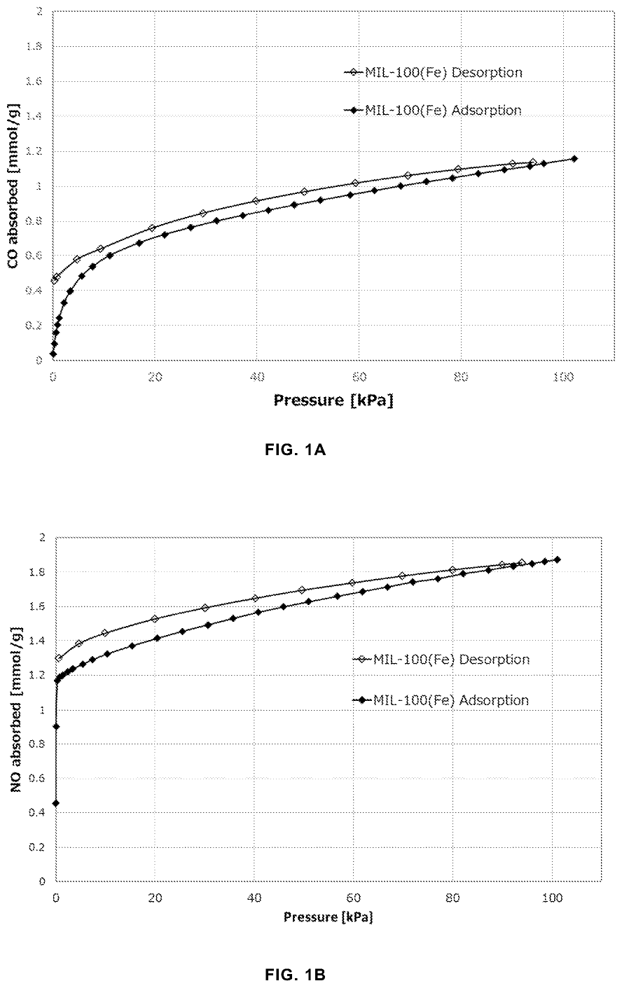

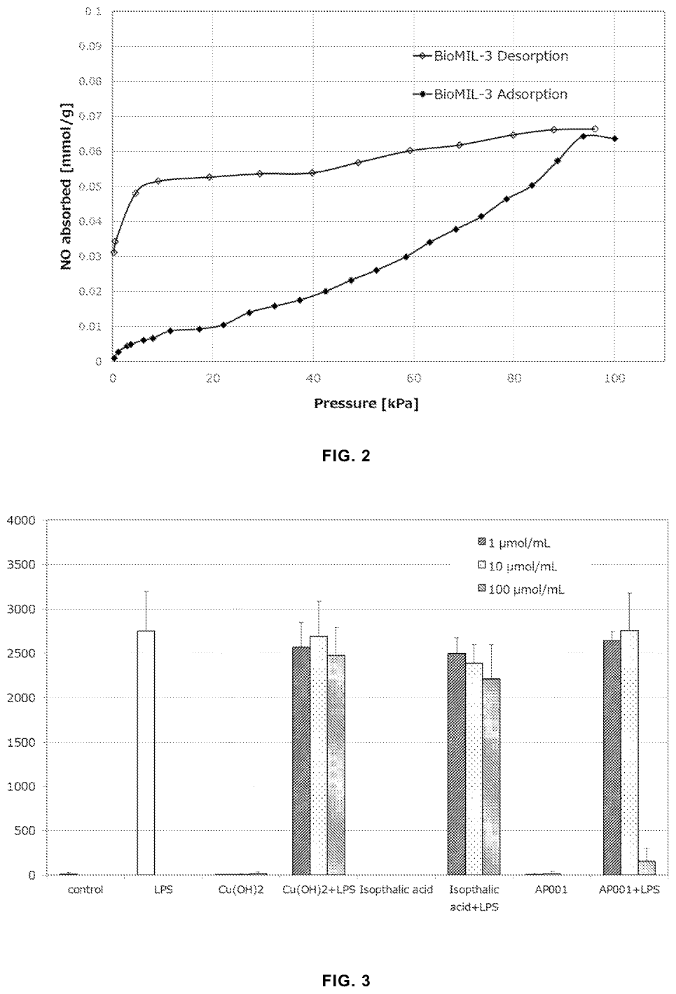

The amount of adsorption was measured by BELSORP-max12 (MicrotracBEL Co., Ltd.). The MOFs in powder form were used for the measurements. Some of the results are shown in FIG. 1A, FIG. 1B and FIG. 2 as representative examples. FIG. 1A is a CO adsorption profile of AP004 [MIL-100 (Fe)]. FIG. 1B is a NO adsorption profile of AP004 [MIL-100 (Fe)]. FIG. 2 is a NO adsorption profile of AP104 (BioMIL-3). In these examples, the adsorption/desorption profiles were irreversible. That is, when seen at the same pressure, the guest amount at the time of desorption was larger than the guest amount at the time of adsorption. Also, the residual amount of the guest in the MOFs were non-zero after performing the adsorption process from a vacuum state to a pressurized state and then performing the desorption process from the pressurized state to the vacuum state.

[Introduction of Immune Signal Transducers into MOFs]

In some of the examples below, the MOFs to which an immune signal transducer had been introduced were employed. Specifically, the degassing was performed by heating the MOF under a nitrogen flow. The sample was then returned to a room temperature and was exposed to an immune signal transducer. In particular, when the immune signal transducer was a gas, the sample returned to room temperature was exposed to a gas flow. A nitrogen flow was then performed at room temperature to discharge excess immune signal transducer. In this way, a MOF compound to which an immune signal transducer had been introduced was obtained.

The existence of the immune signal transducer in the MOF was checked by heating the sample under nitrogen flow and detecting the released immune signal transducer by a detector tube. It was thus confirmed that the immune signal transducer had effectively been introduced into the MOFs.

[Measurement of Cytokine Production Using Mouse-Derived Peritoneal Macrophages (ELISA Method)]

2 mL of 4% thioglycolic acid medium (Difco Laboratories) was administered to a C57BL/6 mouse (7-week-old female), and its peritoneal macrophages were collected. 100 μL of peritoneal macrophages were added to each well of a 96-well plate with a concentration of 1×10⁵ cells/well. 100 μL each of the sample solutions diluted with RPMI medium (100 μg/mL) was added to each well and incubated for 24 hours. 50 μL/well of the supernatant of the cell culture was collected for an evaluation by an ELISA kit (Quantikine ELISA kit, R&D Systems) that corresponds to mouse IL-6, mouse IL-1β, or mouse TNF-α. The tests were conducted six times, and the average and the standard deviation were calculated.

First, the present inventors compared the case where a MOF had been used with the case where only a metal or a ligand had been used. The compositions are summarized in Table 17 below. In the table, MOF means a Metal Organic Framework, LPS means a lipopolysaccharide (Salmonella Minnesota R595) that was added as a positive control, and Gly means glycerin. The measurement results of IL-6 production are shown in FIG. 3.

TABLE 17

| MOF | | | LPS | | | Cell | |
|---|---|---|---|---|---|---|---|
| | Concentration | Concentration | Concentration | | Amount | Concentration | Evaluated |
| Name | [μmol/mL] | [μg/mL] | [ng/mL] | Solvent | [μL/well] | [cells/well] | Value |
| — | — | — | — | Gly | 200 | 1 × 10⁵ | IL-6 |
| | — | — | 100 | | | | |
| Cu(OH)₂ | 1 | 0.98 | — | | | | |
| | 10 | 9.8 | | | | | |
| | 100 | 98 | | | | | |
| | 1 | 0.98 | 100 | | | | |
| | 10 | 9.8 | | | | | |
| | 100 | 98 | | | | | |
| H₂IPA | 1 | 1.66 | — | | | | |
| | 10 | 16.6 | | | | | |
| | 100 | 166 | | | | | |
| | 1 | 1.66 | 100 | | | | |
| | 10 | 16.6 | | | | | |
| | 100 | 166 | | | | | |
| AP001 | 1 | 2.28 | — | | | | |
| | 10 | 22.8 | | | | | |
| | 100 | 228 | | | | | |
| | 1 | 2.28 | 100 | | | | |
| | 10 | 22.8 | | | | | |
| | 100 | 228 | | | | | |

IPA: Isophtalic acid

As shown in FIG. 3, there was a significant difference in IL-6 production between the case where the MOF had been used and the case where only the metal or the ligand had been used. In particular, a large immunosuppressive effect was observed when the MOF had been used at a high concentration.

Next, the present inventors measured the amount of each cytokine produced when the other MOFs had been used. The compositions are summarized in Tables 18 to 22 below. In some examples, MOFs adsorbed with an immune signal transducer were used.

TABLE 18

| MOF | | | | | LPS | | | Cell | |
|---|---|---|---|---|---|---|---|---|---|
| | | Molecular | Concentration | Concentration | Concentration | | Amount | Concentration | Evaluated |
| Name | | Weight | [μmol/mL] | [μg/mL] | [ng/mL] | Solvent | [μL/well] | [cells/well] | Value |
| — | | | — | — | — | Gly | 200 | 1 × 10⁵ | TNF-α |
| | | | — | — | 100 | | | | IL-1β |
| AP008 | Zn(2-methylimidazole)₂ | 229 | 1 | 2 | — | | | | IL-6 |
| ZIF-8 | | | 10 | 23 | | | | | |
| | | | 100 | 229 | | | | | |
| | | | 1 | 2 | 100 | | | | |
| | | | 10 | 23 | | | | | |
| | | | 100 | 229 | | | | | |
| AP004 | Fe₂O(OH)(BTC)₂ | 615 | 1 | 6 | — | | | | |
| MIL- | | | 10 | 62 | | | | | |
| 100(Fe) | | | 100 | 615 | | | | | |
| | | | 1 | 6 | 100 | | | | |
| | | | 10 | 62 | | | | | |
| | | | 100 | 615 | | | | | |
| AP006 | Al(OH)(fumarate) | 158 | 1 | 2 | — | | | | |
| Al(Fumarate) | | | 10 | 16 | | | | | |
| | | | 100 | 158 | | | | | |
| | | | 1 | 2 | 100 | | | | |
| | | | 10 | 16 | | | | | |
| | | | 100 | 158 | | | | | |
| AP005 | Al(OH)(BDC) | 295 | 1 | 3 | — | | | | |
| MIL- | | | 10 | 30 | | | | | |
| 53(Al) | | | 100 | 295 | | | | | |
| | | | 1 | 3 | 100 | | | | |
| | | | 10 | 30 | | | | | |
| | | | 100 | 295 | | | | | |

BTC: Trimesic acid
BDC: Terephthalic acid

TABLE 19

| MOF | | | | | LPS | | | Cell | |
|---|---|---|---|---|---|---|---|---|---|
| | | Molecular | Concentration | Concentration | Concentration | | Amount | Concentration | Evaluated |
| Name | | Weight | [μmol/mL] | [μg/mL] | [ng/mL] | Solvent | [μL/well] | [cells/well] | Value |
| — | | | — | — | — | Gly | 200 | 1 × 10⁵ | TNF-α |
| | | | — | — | 100 | | | | IL-1β |
| AP015 | Ca(Malate) | 174 | 1 | 2 | — | | | | IL-6 |
| | | | 10 | 17 | | | | | |
| | | | 100 | 174 | | | | | |
| | | | 1 | 2 | 100 | | | | |
| | | | 10 | 17 | | | | | |
| | | | 100 | 174 | | | | | |
| AP104 | Ca₂(Tazb) | 434 | 1 | 4 | — | | | | |
| BioMIL-3 | | | 10 | 43 | | | | | |
| | | | 100 | 434 | | | | | |
| | | | 1 | 4 | 100 | | | | |
| | | | 10 | 43 | | | | | |
| | | | 100 | 434 | | | | | |
| AP009 | Mg₂(Formate)₅ | 114 | 1 | 1 | — | | | | |
| Mg(Formate) | | | 10 | 11 | | | | | |
| | | | 100 | 114 | | | | | |
| | | | 1 | 1 | 100 | | | | |
| | | | 10 | 11 | | | | | |
| | | | 100 | 114 | | | | | |
| AP014 | La(BTB) | 574 | 1 | 6 | — | | | | |
| | | | 10 | 57 | | | | | |
| | | | 100 | 574 | | | | | |
| | | | 1 | 6 | 100 | | | | |
| | | | 10 | 57 | | | | | |
| | | | 100 | 574 | | | | | |

Tazb:3,3′,5,5′-Azobenzene tetracarboxylic acid
BTB: 1,3,5-Tris(4-carboxyphenyl)benzene

TABLE 20

| MOF | | | | | LPS | | | Cell | |
|---|---|---|---|---|---|---|---|---|---|
| | | Molecular | Concentration | Concentration | Concentration | | Amount | Concentration | Evaluated |
| Name | | Weight | [μmol/mL] | [μg/mL] | [ng/mL] | Solvent | [μL/well] | [cells/well] | Value |
| — | | | — | — | — | Gly | 200 | 1 × 10⁵ | TNF-α |
| | | | — | — | 100 | | | | IL-1β |
| AP003 | Fe(BTC) | 263 | 1 | 3 | — | | | | IL-6 |
| Fe(BTC) | | | 10 | 26 | | | | | |
| | | | 100 | 263 | | | | | |
| | | | 1 | 3 | 100 | | | | |
| | | | 10 | 26 | | | | | |
| | | | 100 | 263 | | | | | |
| AP102 | Ca(CPP)•H₂O | 258.18 | 1 | 3 | — | | | | |
| | | | 10 | 26 | | | | | |
| | | | 100 | 258 | | | | | |
| | | | 1 | 3 | 100 | | | | |
| | | | 10 | 26 | | | | | |
| | | | 100 | 258 | | | | | |
| AP103 | Ca(Zol)-H₂O | 329.17 | 1 | 3 | — | | | | |
| | | | 10 | 33 | | | | | |
| | | | 100 | 329 | | | | | |
| | | | 1 | 3 | 100 | | | | |
| | | | 10 | 33 | | | | | |
| | | | 100 | 329 | | | | | |
| AP106 | Mg(Mino)₂•3H₂O | 720.6 | 1 | 7 | — | | | | |
| | | | 10 | 72 | | | | | |
| | | | 100 | 721 | | | | | |
| | | | 1 | 7 | 100 | | | | |
| | | | 10 | 72 | | | | | |
| | | | 100 | 721 | | | | | |

BTC: Trimesic acid
Tazb:3,3′,5,5′-Azobenzene tetracarboxylic acid

TABLE 21

| MOF | | | | | | LPS | | | | |
|---|---|---|---|---|---|---|---|---|---|---|
| | | Immune | | Con- | Con- | Con- | | | Cell | |
| | | Signal | Molecular | centration | centration | centration | | Amount | Concentration | Evaluated |
| Name | | Transducer | Weight | [μmol/mL] | [μg/mL] | [ng/mL] | Solvent | [μL/well] | [cells/well] | Value |
| — | | | | — | — | — | Gly | 200 | 1 × 10⁵ | TNF-α |
| | | | | — | — | 100 | | | | IL-1β |
| AP104 | Ca(Tazb) | NO | 434 | 1 | 4 | — | | | | IL-6 |
| BioMIL-3 | | | | 10 | 43 | | | | | |
| | | | | 100 | 434 | | | | | |
| | | | | 1 | 4 | 100 | | | | |
| | | | | 10 | 43 | | | | | |
| | | | | 100 | 434 | | | | | |
| AP004 | Fe₃O(OH)(BTC)₂ | NO | 679 | 1 | 7 | — | | | | |
| MIL-100(Fe) | | | | 10 | 68 | | | | | |
| | | | | 100 | 679 | | | | | |
| | | | | 1 | 7 | 100 | | | | |
| | | | | 10 | 68 | | | | | |
| | | | | 100 | 679 | | | | | |
| AP004 | Fe₃O(OH)(BTC)₂ | CO | 679 | 1 | 7 | — | | | | |
| MIL-100(Fe) | | | | 10 | 68 | | | | | |
| | | | | 100 | 679 | | | | | |
| | | | | 1 | 7 | | | | | |
| | | | | 10 | 68 | 100 | | | | |
| | | | | 100 | 679 | | | | | |
| AP004 | Fe₃O(OH)(BTC)₂ | O₂ | 679 | 1 | 7 | — | | | | |
| MIL-100(Fe) | | | | 10 | 68 | | | | | |
| | | | | 100 | 679 | | | | | |
| | | | | 1 | 7 | 100 | | | | |
| | | | | 10 | 68 | | | | | |
| | | | | 100 | 679 | | | | | |
| AP107 | Al₂(PBA)₂ | — | 671 | 1 | 7 | — | | | | |
| Al(PBA) | | | | 10 | 67 | | | | | |
| | | | | 100 | 671 | | | | | |
| | | | | 1 | 7 | 100 | | | | |
| | | | | 10 | 67 | | | | | |
| | | | | 100 | 671 | | | | | |
| AP108 | Ca(Tartrate) | — | 188 | 1 | 2 | — | | | | |
| Ca(Tartrate) | | | | 10 | 19 | | | | | |
| | | | | 100 | 188 | | | | | |
| | | | | 1 | 2 | 100 | | | | |
| | | | | 10 | 19 | | | | | |
| | | | | 100 | 188 | | | | | |

BTC: Trimesic acid
Tazb:3,3′,5,5′-Azobenzene tetracarboxylic acid

TABLE 22

| MOF | | | | | | LPS | | | Cell | |
|---|---|---|---|---|---|---|---|---|---|---|
| | | Immune | | Con- | Con- | Con- | | | Con- | |
| | | Signal | Molecular | centration | centration | centration | | Amount | centration | Evaluated |
| Name | | Transducer | Weight | [μmol/mL] | [μg/mL] | [ng/mL] | Solvent | [μL/well] | [cells/well] | Value |
| — | | | | — | — | — | Gly | 200 | 1 × 10⁵ | TNF-α |
| | | | | — | — | 100 | | | | IL-1β |
| Ni-MOF-74 | Ni(C₂H₂O₂) | NO | 257 | 1 | 3 | — | | | | IL-6 |
| | | | | 10 | 26 | | | | | |
| | | | | 100 | 257 | | | | | |
| | | | | 1 | 3 | 100 | | | | |
| | | | | 10 | 26 | | | | | |
| | | | | 100 | 257 | | | | | |
| Ni-MOF-74 | Ni(C₂H₂O₂) | NO | 257 | 1 | 3 | — | | | | |
| | | | | 10 | 26 | | | | | |
| | | | | 100 | 257 | | | | | |
| | | | | 1 | 3 | 100 | | | | |
| | | | | 10 | 26 | | | | | |
| | | | | 100 | 257 | | | | | |
| Co-MOF-74 | Co(C₂H₂O₂) | — | 257 | 1 | 3 | — | | | | |
| | | | | 10 | 26 | | | | | |
| | | | | 100 | 257 | | | | | |
| | | | | 1 | 3 | | | | | |
| | | | | 10 | 26 | 100 | | | | |
| | | | | 100 | 257 | | | | | |
| Co-MOF-74 | Co(C₂H₂O₂) | NO | 257 | 1 | 3 | — | | | | |
| | | | | 10 | 26 | | | | | |
| | | | | 100 | 257 | | | | | |
| | | | | 1 | 3 | 100 | | | | |
| | | | | 10 | 26 | | | | | |
| | | | | 100 | 257 | | | | | |
| MIL-BB-A | Fe(C₂H₂O₂) | — | 172 | 1 | 2 | — | | | | |
| | | | | 10 | 17 | | | | | |
| | | | | 100 | 172 | | | | | |
| | | | | 1 | 2 | 100 | | | | |
| | | | | 10 | 17 | | | | | |
| | | | | 100 | 172 | | | | | |
| MIL-BB-A | Fe(C₂H₂O₂) | NO | 172 | 1 | 2 | — | | | | |
| | | | | 10 | 17 | | | | | |
| | | | | 100 | 172 | | | | | |
| | | | | 1 | 2 | 100 | | | | |
| | | | | 10 | 17 | | | | | |
| | | | | 100 | 172 | | | | | |
| MIL-BB-B | Fe(C₂H₂O₂) | — | 222 | 1 | 2 | — | | | | |
| | | | | 10 | 22 | | | | | |
| | | | | 100 | 222 | | | | | |
| | | | | 1 | 2 | 100 | | | | |
| | | | | 10 | 22 | | | | | |
| | | | | 100 | 222 | | | | | |
| MIL-BB-B | Fe(C₂H₂O₂) | NO | 222 | 1 | 2 | — | | | | |
| | | | | 10 | 22 | | | | | |
| | | | | 100 | 222 | | | | | |
| | | | | 1 | 2 | 100 | | | | |
| | | | | 10 | 22 | | | | | |
| | | | | 100 | 222 | | | | | |

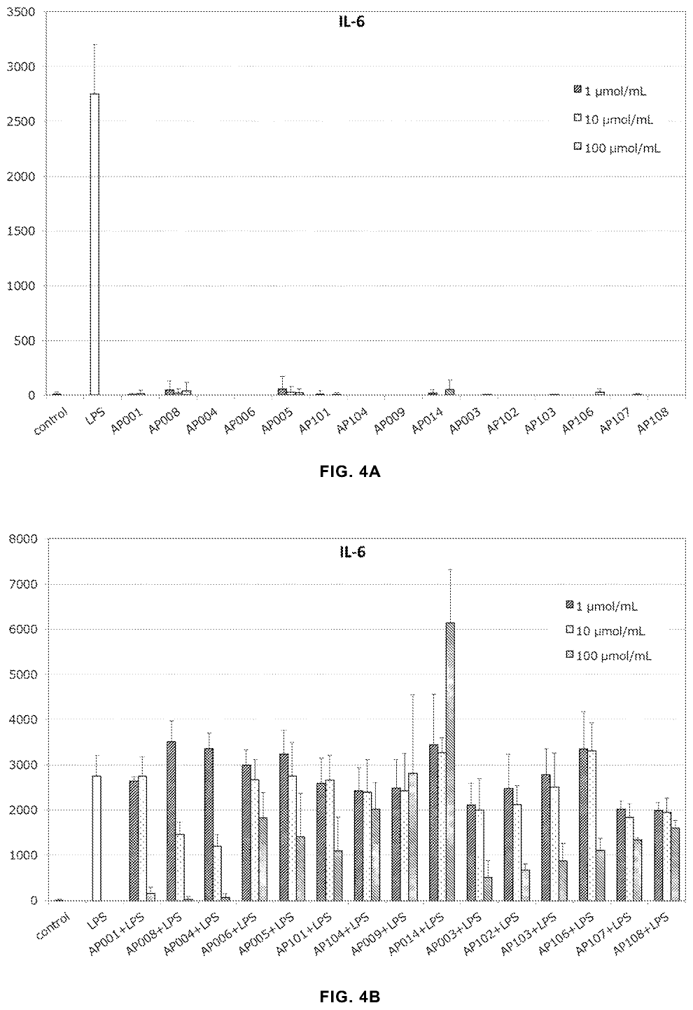

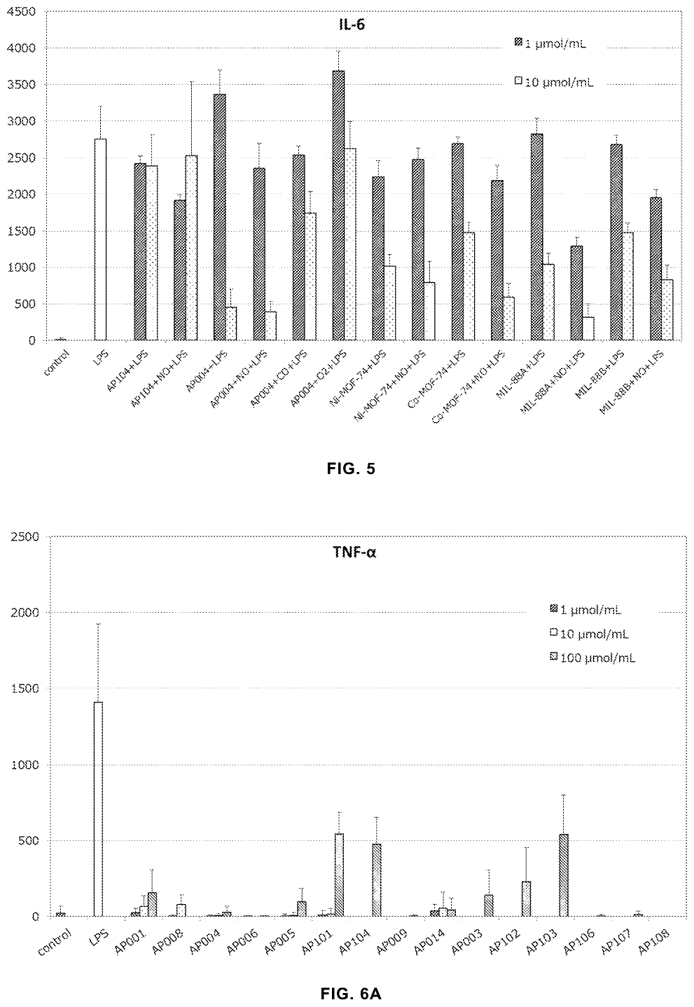

FIGS. 4A and 4B show the measurement results of IL-6 production. FIG. 5 shows the measurement results of IL-6 production when a gas component is included as an immune signal transducer.

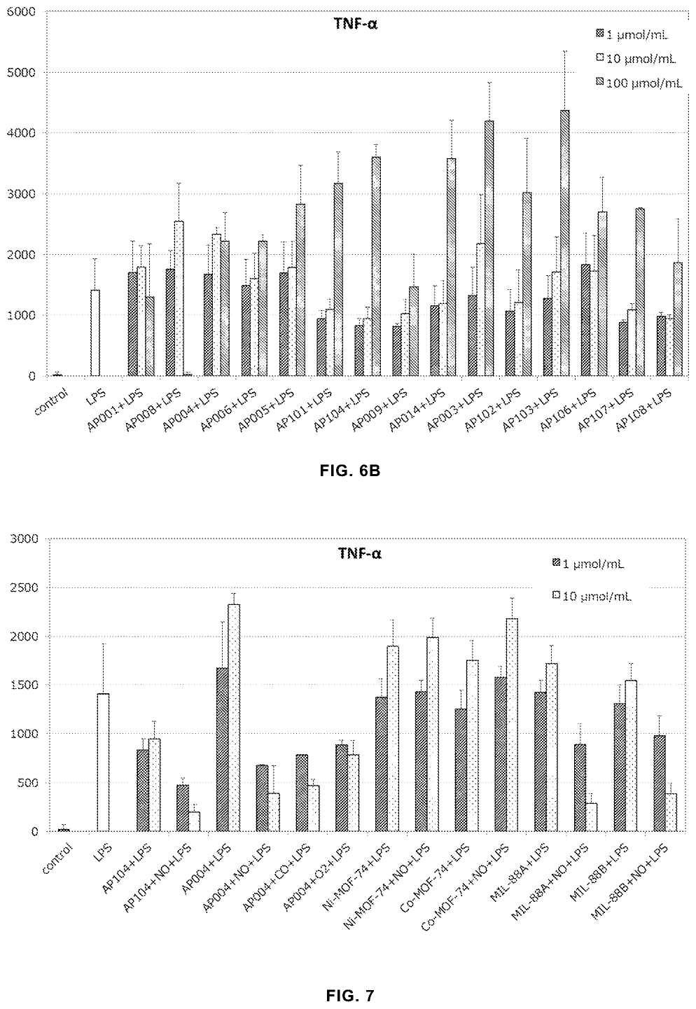

FIGS. 6A and 6B show the measurement results of TNF-α production. FIG. 7 shows the measurement results of the TNF-α production when a gas component is included as an immune signal transducer.

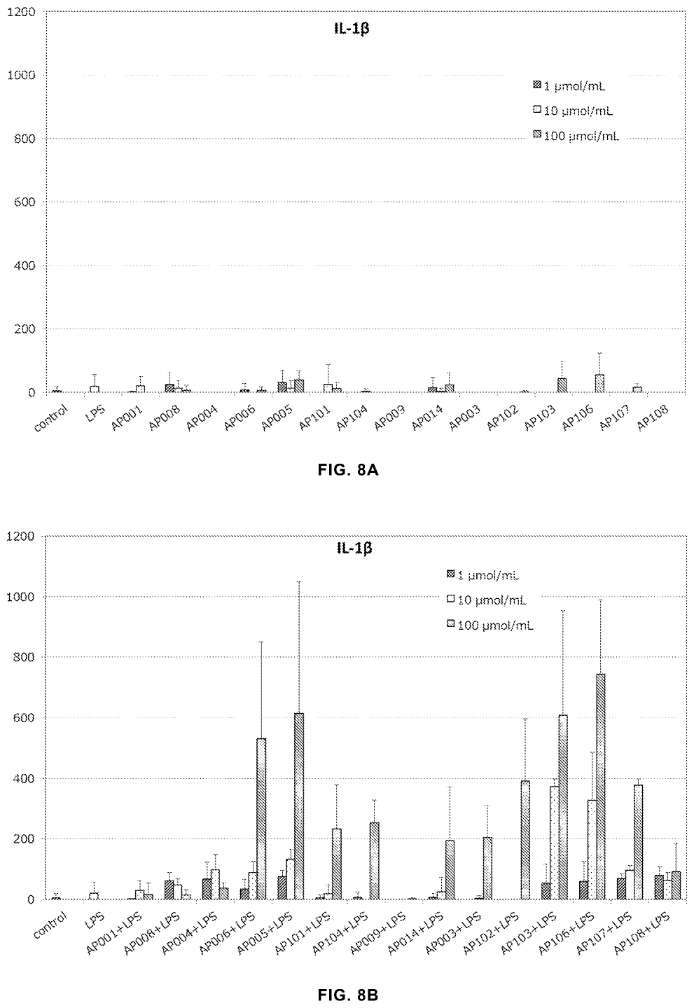

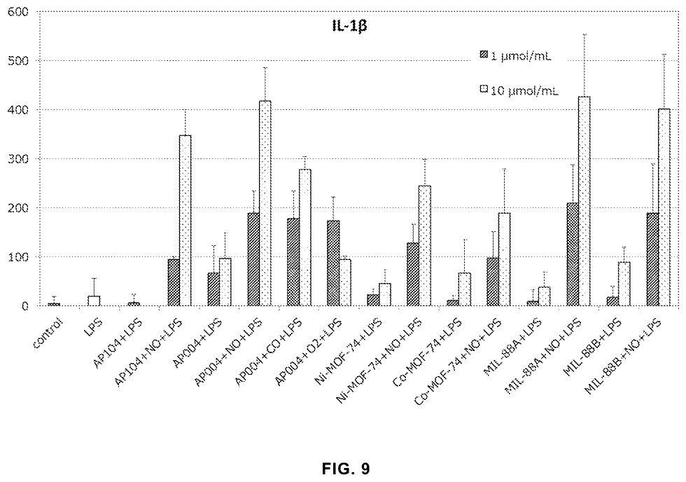

FIGS. 8A and 8B show the measurement results of IL-1β production. FIG. 9 shows the measurement results of IL-1β production when a gas component is included as an immune signal transducer.

Tables 23 and 24 below summarize the results qualitatively. As can be seen from the results, it was shown that the immune function can be adjusted by use of the MOFs. It was also shown that the immune function can be additionally regulated by further introducing a gas component as an immune signal transducer.

TABLE 23

| MOF | | IL-6 | TNF-α | IL-1β |
|---|---|---|---|---|
| AP001 | MODOKI | ↓↓ | | |
| AP008 | ZIF-8 | ↓↓ | ↓↓ | |
| AP004 | MIL-100(Fe) | ↓↓ | | |
| AP006 | Al(Fumarate) | | | ↑↑ |
| AP005 | MIL-53(Al) | | ↑ | ↑↑ |
| AP101 | Ca(Malate) | | ↑ | ↑ |
| AP104 | BioMIL-3 | | ↑↑ | ↑ |
| AP009 | Mg(Formate) | | | |
| AP014 | MIL-103(La) | ↑↑ | ↑ | ↑ |
| AP003 | Fe-BTC | ↓ | ↑↑ | ↑ |
| AP102 | Ca₃(PBA)₂ | ↓ | ↑ | ↑ |
| AP103 | Ca(Zoledronate) | ↓ | ↑↑ | ↑↑ |
| AP106 | Mg(Minodronate) | | ↑ | ↑↑ |
| AP107 | Al₂(PBA)₃ | | ↑ | ↑ |
| AP108 | Ca(Tartrate) | | | |
| — | Ni-MOF-74 | ↓ | ↑ | |
| — | Co-MOF-74 | ↓ | ↑ | |
| — | MIL-88A | ↓ | | |
| — | MIL-88B | ↓ | | |

TABLE 24

| | | Immune Signal | | | |
|---|---|---|---|---|---|
| MOF | | Transducer | IL-6 | TNF-α | IL-1β |
| AP004 | MIL-100(Fe) | NO | ↓↓ | ↓ | ↑↑ |
| | | CO | ↓ | ↓ | ↑ |
| | | O₂ | ↑ | ↓ | ↑ |
| AP104 | BioMIL-3 | NO | | ↓ | ↑↑ |
| — | Ni-MOF-74 | NO | ↓ | ↑ | ↑ |
| — | Co-MOF-74 | NO | ↓ | ↑ | ↑ |
| — | MIL-88A | NO | ↓↓ | ↓ | ↑↑ |
| — | MIL-88B | NO | ↓ | ↓ | ↑↑ |

1. A pharmaceutical composition for a disease related to immunity, comprising a Metal Organic Framework (MOF).

2. The pharmaceutical composition according to claim 1, further comprising an immune signal transducer.

3. The pharmaceutical composition according to claim 1, wherein at least a part of the immune signal transducer is contained in pores of the MOF.

4. The pharmaceutical composition according to claim 3, wherein the MOF is configured to decompose in vivo to release at least a part of the immune signal transducer.

5. The pharmaceutical composition according to claim 2, wherein the immune signal transducer is a small molecule having a molecular weight of 1000 or less.

6. The pharmaceutical composition according to claim 5, wherein the immune signal transducer is a gas at 25° C. and 100 kPa.

7. The pharmaceutical composition according to claim 2, wherein the immune signal transducer is a factor that is configured to act on keratinocytes, monocytes, lymphocytes, or granulocytes.

8. The pharmaceutical composition according to claim 1, wherein the MOF comprises at least one metal element selected from the group consisting of calcium, magnesium, iron, zinc, aluminum, potassium, and sodium.

9. The pharmaceutical composition according to claim 1, wherein the pharmaceutical composition is configured to be administered by an oral administration, a transdermal administration, and/or a mucosal administration.

10. The pharmaceutical composition according to claim 1, wherein the pharmaceutical composition is configured to be administered by an intradermal injection, a subcutaneous injection, or an intramuscular injection.

11. The pharmaceutical composition according to claim 3, wherein the immune signal transducer is a small molecule having a molecular weight of 1000 or less.

12. The pharmaceutical composition according to claim 4, wherein the immune signal transducer is a small molecule having a molecular weight of 1000 or less.

13. The pharmaceutical composition according to claim 11, wherein the immune signal transducer is a gas at 25° C. and 100 kPa.

14. The pharmaceutical composition according to claim 12, wherein the immune signal transducer is a gas at 25° C. and 100 kPa.

15. The pharmaceutical composition according to claim 2, wherein the MOF comprises at least one metal element selected from the group consisting of calcium, magnesium, iron, zinc, aluminum, potassium, and sodium.

16. The pharmaceutical composition according to claim 3, wherein the MOF comprises at least one metal element selected from the group consisting of calcium, magnesium, iron, zinc, aluminum, potassium, and sodium.

17. The pharmaceutical composition according to claim 4, wherein the MOF comprises at least one metal element selected from the group consisting of calcium, magnesium, iron, zinc, aluminum, potassium, and sodium.

18. The pharmaceutical composition according to claim 5, wherein the MOF comprises at least one metal element selected from the group consisting of calcium, magnesium, iron, zinc, aluminum, potassium, and sodium.

19. The pharmaceutical composition according to claim 6, wherein the MOF comprises at least one metal element selected from the group consisting of calcium, magnesium, iron, zinc, aluminum, potassium, and sodium.

20. The pharmaceutical composition according to claim 7, wherein the MOF comprises at least one metal element selected from the group consisting of calcium, magnesium, iron, zinc, aluminum, potassium, and sodium.
